(19) United States
(12) Patent Application Publication
(10) Pub. No.: US 2013/0224115 A1
(43) Pub. Date: Aug. 29, 2013

(54) NON-RADIOACTIVE AGENTS FOR NEUROBLASTOMA IMAGING

(71) Applicants: Wei Wang, Houston, TX (US); Jason Shohet, Houston, TX (US); Michel Mawad, Houston, TX (US); Shi Ke, Missouri City, TX (US)

(72) Inventors: Wei Wang, Houston, TX (US); Jason Shohet, Houston, TX (US); Michel Mawad, Houston, TX (US); Shi Ke, Missouri City, TX (US)

(73) Assignee: Baylor College of Medicine, Houston, TX (US)

(21) Appl. No.: 13/638,060

(22) PCT Filed: Apr. 1, 2011
(86) PCT No.: PCT/US11/30868

§ 371 Date: May 9, 2013

Related U.S. Application Data
(60) Provisional application No. 61/320,129, filed on Apr. 1, 2010.

(51) Int. Cl. C07D 209/26

(52) U.S. Cl. CPC C07D 209/26
USPC 424/9.1; 548/455

(57) ABSTRACT

Multi-modal tumor imaging of neuroblastoma is essential for tumor staging, response evaluation and detection of relapsed diseased. The present invention provides a norepinephine analogue with a near-infrared (near-IR) dye, W765-BG, that efficiently and stably detects neuroblastoma in vivo using near-IR optical imaging. Confocal microscopy and optical imaging of neuroblastoma xenografts shows cell specific uptake and reveals exceptional tumor retention with a high tumor-to-tissue ratio up to 7 days after injection of W765-BG.

### CROSS-REFERENCE TO RELATED APPLICATIONS

This application is a national phase filing under USC §371 from PCT Application Serial No. PCT/US2011/030868, filed Apr. 1, 2011, which claims the benefit of U.S. Provisional Application Ser. No. 61/320,129, filed Apr. 1, 2010, both of which applications are hereby incorporated by reference in their entirety.

### STATEMENT REGARDING FEDERALLY SPONSORED RESEARCH OR DEVELOPMENT

This invention was made with government support under K08-CA09517 awarded by the National Institutes of Health (NIH). The government has certain rights in the invention.

### TECHNICAL FIELD

The present disclosure relates generally to imaging agents and, in particular, imaging agents used to image and/or detect neuroblastoma. More specifically, the present disclosure concerns the imaging of neuroblastoma using a nonradioactive near infrared dye labeled benzylguanidine analog.

### BACKGROUND OF THE INVENTION

Neuroblastoma is the most common extra-cranial solid cancer in pediatric patients. Despite chemotherapy, surgery and radiation therapy, neuroblastoma's aggressive malignancy accounts for more than 15% of all pediatric cancer deaths (Lonergan et al., 2002 and Maris et al., 2007). Metastatic spread is the most important risk factor in predicting survival. Other risk factors are the age of the child at diagnosis and the biologic features of the tumor. Survival for localized tumors nears 95% with surgical resection alone. However, treatment for metastatic or biologically aggressive tumors (high risk neuroblastoma) requires an intense multi-modality approach including surgical resection, chemotherapy, radiotherapy, and high dose chemotherapy with autologous stem cell rescue. Despite initial responses in the majority of patients with high risk neuroblastoma, the tumor commonly recurs. Effectively, treating patients with neuroblastomas remains a challenging task for both clinicians and researchers.

Neuroblastoma's arise from neural crest precursors that express components of the mature catecholamine metabolic pathway. This catecholamine-secreting tumor is derived from neural crest cells, which are precursors of the sympathetic nervous system (Rha et al., 2003). Ninety to 95% of tumors actively take up norepinephrine precursors via the specific norepinephrine transporter and non-specific pathways. MIBG (meta-iodobenzylguanidine) is a norepinephrine analog that utilizes these transporter pathways. While MIBG has been used clinically for over 2 decades, the exact mechanism of MIBG accumulation in human neuroblastoma cells and tumors is not well defined (Koopmans et al., 2009). MIBG and other norepinephrine analogues are taken up by neuroendocrine derived tumors such as neuroblastoma and pheochromocytoma through multiple mechanisms including active transport by amine precursor receptors such as the NET (Norepinehrine receptor) and non-specific metabolic uptake. Although MIBG negative tumors have been found to be NET negative, the correlation between level of NET expression and MIBG uptake remains poorly defined for neuroblastoma. There is evidence that the vesicular monoamine transporters (VMAT1 and VMAT2), which are highly expressed in neuroblastoma, act to sequester MIBG in cytoplasmic granules (Kolby et al., 2003).

Neuroblastoma tumors can develop anywhere in the sympathetic nervous system with variable signs and symptoms in young children (Papaioannou and McHugh, 2005). Therefore, a complete understanding of specific characteristics of the disease, including tumor location, size, stage, early detection of relapse and treatment response, is critical for designing effective treatment regimens. Molecular imaging technology plays an important role in this process, because imaging may be used as a tool to interrogate cellular and molecular biological events.

The most commonly used neuroblastoma imaging agent is meta-iodobenzylguanidine (MIBG), labeled with an iodine radioisotope, I¹²³ or I¹³¹. Radiolabeling MIBG with I¹²³ emits low energy (159 Kev) gamma radiation which is ideal for single photon emission computed tomography (SPECT) detection and makes MIBG radiolabeled with I¹²³ useful for diagnosis and management of neuroblastoma. MIBG radiolabeled with I¹²³ has been used in clinical practice since the 1980s (Valk et al., 1981; Wieland et al., 1980), and has been a mainstay in imaging neuroblastomas for decades in the pediatric population (Howman-Giles et al., 2007; Rufini et al., 2006; Vik et al., 2009).

However, long-term safety concerns associated with the use of radiolabeled meta-iodobenzylguanidine are valid, especially when it is used repeatedly in young patients, as radiation exposure increases the risk of developing secondary cancers and increased therapy-related toxicity (Howman-Giles et al., 2007). Another health concerns is that by exposing the patient to radioactive iodine adds to the logistical complications by posing a risk to the thyroid which is abrogated by the use of concomitant oral iodine.

Radioactive compounds are limited for longitudinal and cell studies due to their relatively short half-life (Sisson and Shulkin, 1999; Wafelman et al., 1994). The very short T_{1/2} of I¹²³ is about 12.5 hours. This relatively short half-life requires that the agent is used within a day of synthesis for maximum sensitivity and limits qualitative comparison between scans in the same patient performed at different times.

Another limitation is that radioactive compounds are limited by their low resolution (Wong and Kim, 2009). The spatial resolution of SPECT images derived from radiolabeled MIBG is poor thereby adding little anatomic information. False negatives from extensive necrotic tumors, drug interference (e.g. labetalol) or technical problems are common as well as false positives from thymus and brown fat. Cardiac uptake, liver uptake or uptake in nonspecific and non-uniform bowel may contribute to reducing the sensitivity and specificity MIBG scans. As such, the disadvantages of radioactive agents restrict their use in both pre-clinical and clinical investigations.

Optical imaging is an active and promising area for both in vitro and in vivo molecular imaging studies. Of the various optical imaging techniques used to date, near infrared (NIR) fluorescence imaging is particularly promising. The wavelength for near infrared light ranges from 700 to 900 nanometers with minimal autofluorescence, and is minimally absorbed by hemoglobin (the principal absorber of visible light), water, and lipids (the principal absorbers of infrared light). Considering the advantages of NIR imaging, this method could provide an attractive approach for improving the imaging accuracy and safety for pediatric patients.

The object of this disclosure is to provide nonradioactive NIR optical imaging agents based upon the structure of MIBG. The nonradioactive NIR optical imaging agent, W765-BG, has been evaluated at the cellular level by confocal microscopy, and in vivo in a whole animal using a human neuroblastoma xenograft model. The specific uptake of this agent in both neuroblastoma cells and tumors demonstrate that W765-BG is useful for neuroblastoma studies and diagnoses.

### BRIEF SUMMARY OF THE INVENTION

The present invention is directed to a composition and method for non-radioactive, near-infrared imaging. Specifically, the compositions and methods disclosed herein allow for imaging of neuroblastomas without the use of radio-labeled imaging agents.

Accordingly, the present disclosure provides a composition having a meta-functionalized benzyl guanidine, a spacer moiety, a linker moiety and non-radioactive dye. The spacer moiety has a reactive amino functionality. The spacer moiety is chemically bonded to the meta-functionalized benzyl guanidine, the linker moiety is chemically bonded to the spacer moiety and the dye is chemically bonded to the linker moiety. The chemical bond connecting the meta-functionalized benzyl guanidine and the spacer moiety is an ester, ether, thioether, thioester, amide, a carbon-carbon single bond, a carbon-carbon double bond, or a carbon-carbon triple bond.

The spacer moiety is a functionalized alkyl_{(C=1-8)}, alkenyl_{(C=1-8)}, or aralkyl_{(C=1-8)}, or a substituted version of any of these moieties. The linker moiety is a functionalized alkyl_{(C=1-8)}, alkenyl_{(C=1-8)}, or aralkyl_{(C=1-8)}, or a substituted version of any of these moieties. In some examples, the dye is a contrast agent. In specific examples, the dye is IRdye800CW, IRdye800RS, or IRdye700DX.

In additional and alternate embodiments, the present disclosure provides composition having the general formula:

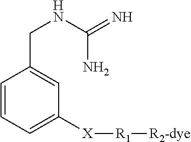

wherein X is O, CH₂, CH, C, NH, S or C═O; R₁ has the general formula

R₃-R₄-R₅

wherein R₃ is chemically bonded to X to form an amide, thioester, ester, ether, carbon-carbon bond, carbon-carbon double bond, or a carbon-carbon triple bond, R₄ is alkyl_{(C=1-8)}, alkenyl_{(C=1-8)}, or aralkyl_{(C=1-8)}, or a substituted version of any of these groups, R₅ is chemically bonded to R₂ to from an amide, thioester, ester, ether, carbon-carbon bond, carbon-carbon double bond, or a carbon-carbon triple bond, R₂ is alkyl_{(C=1-8)}, alkenyl_{(C=1-8)}, or aralkyl_{(C=1-8)}, or a substituted version of any of these groups and forms an amino bond with the dye, and the dye is a fluorophore.

In specific embodiments, X is NH. In other embodiments, R₃ forms an amide bond. In certain embodiments, R₄ is alkyl_{(C=1-2)}. In some embodiments, R₅ and the R₂ are chemically joined by an amide bond. In specific examples, the composition has the formula:

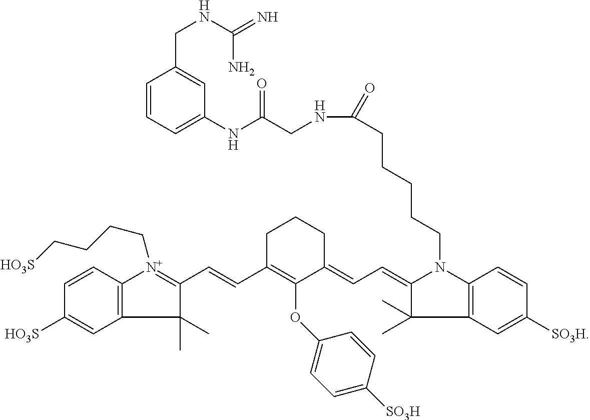 [SMILES: CC1(C)C(/C=C/C2=C(Oc3ccc(S(=O)(=O)O)cc3)C(=C/C=C3/N(CCCCCC(=O)NCC(=O)Nc4cccc(CNC(=N)N)c4)c4ccc(S(=O)(=O)O)cc4C3(C)C)/CCC2)=[N+](CCCCS(=O)(=O)O)c2ccc(S(=O)(=O)O)cc21]

Additionally, the present disclosure provides a method for imaging neuroblastomas comprising the step of treating a subject with a compound having the general formula:

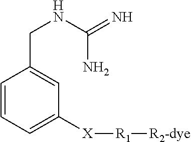

wherein X is O, CH₂, CH, C, NH, S or C═O; R₁ has the general formula

R₃-R₄-R₅

wherein R₃ is chemically bonded to X to form an amide, thioester, ester, ether, carbon-carbon bond, carbon-carbon double bond, or a carbon-carbon triple bond, R₄ is alkyl_{(C=1-8)}, alkenyl_{(C=1-8)}, or aralkyl_{(C=1-8)}, or a substituted version of any of these groups, R₅ is chemically bonded to R₂ to from an amide, thioester, ester, ether, carbon-carbon bond, carbon-carbon double bond, or a carbon-carbon triple bond, R₂ is alkyl_{(C=1-8)}, alkenyl_{(C=1-8)}, or aralkyl_{(C=1-8)}, or a substituted version of any of these groups and forms an amino bond with the dye, and the dye is a fluorophore.

In some embodiments, the particular composition used to image neuroblastomas has the formula:

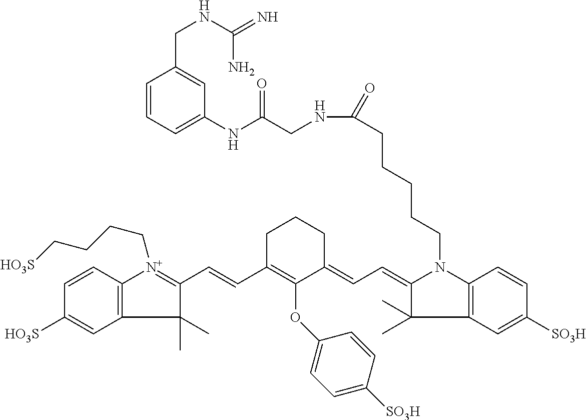 [SMILES: CC1(C)C(/C=C/C2=C(Oc3ccc(S(=O)(=O)O)cc3)C(=C/C=C3/N(CCCCCC(=O)NCC(=O)Nc4cccc(CNC(=N)N)c4)c4ccc(S(=O)(=O)O)cc4C3(C)C)/CCC2)=[N+](CCCCS(=O)(=O)O)c2ccc(S(=O)(=O)O)cc21]

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows the imaging of W765-BG in neuroblastoma cells. Cells treated with W765-BG (top panel) or free dye (bottom panel), and were visualized by confocal microscopy (a) is the cell nuclei, (b) shows the cell with the dye, W765-BG (top panel) and free dye (bottom panel), and (c) shows the merged image of (a) and (b).

FIG. 2 shows the imaging of W765-BG in human tumor xenografts in mice. Mice were injected with NGP.Luc cells, followed by injections with Luciferin and W765-BG. Whole animal images were collected over the course of eight days, and the 48 hour time point is shown in FIG. 2 where FIG. 2A is the white light image, FIG. 2B is the X-ray image, FIG. 2C is the NIR image, FIG. 2D is the luciferase image, FIG. 2E is the merged image of FIG. 2C and FIG. 2D, and FIG. 2F is the merged image of FIG. 2C, FIG. 2D, and FIG. 2E.

FIG. 3 shows the confocal images of W765-BG uptake in human neuroblastoma cells. FIG. 3A shows bright field images of cell morphology and location. FIG. 3B shows the cell nuclei from a signal cell stack at a thickness of 0.5 micrometers. FIG. 3C shows the W765-BG signal from the image of FIG. 3B. FIG. 3D shows the merged images of FIGS. 3A-C. The results show that the cells maintain their morphology after incubation with W765-BG overnight. The cell nuclei signals are from inside the cell membrane. W765-BG binds to all cells and incorporates into the cell nuclei (yellow). FIG. 3E shows a high magnification overlaid view of W765-BG uptake by a neuroblastoma cell. FIG. 3F shows a high magnification merged image of cell morphology and free dye uptake. FIG. 3G shows that W765-BG signal intensity comes from the cell. FIG. 3H shows the free dye signal intensity. FIG. 3 shows that the neuroblastoma cells take up the W765-BG agent, but not the free dye.

FIG. 4 shows in vivo images of neuroblastoma xenografts. FIG. 4A shows the luciferase image of NB1691.Luc xenograft. The tumor node is localized with clear tumor margins at this stage. FIG. 4B shows a vasculature image of NBG169.Luc tumor xenograft. A black hole is found in the region indicated by the arrow. The tumor-to-background ratio of this region is 0.79. FIG. 4C shows the W765-BG image of the same animal. A signal decrease region is indicated by the arrow. The tumor-to-background ratio of this region is 0.95. FIG. 4D shows a merged luciferase, vasculature and W765-BG image. The luciferase positive tumor nodal fits perfectly into the black hole of the vasculature and signal decrease region of W765-BG images. FIG. 4E shows merged X-ray and W765-BG images with anatomical location of whole body W765-BG signal distribution. FIG. 4F shows merged multi-energy images indicating the relationship between tumor node, vasculature, W765-BG, and anatomy. FIG. 4G shows a luciferase image of NGP.luc xenograft. The tumor growth pattern is diffused and without a clear margin compared to the NB1691.Luc tumor. FIG. 4H shows a vasculature image with an increased tumor to background ratio of 1.13 (TBR=1.13). FIG. 4I shows a WW765-BG signal slightly increased at this stage (TBR=1.08). FIG. 4J shows merged images of the NGP.Luc tumor having a different growth pattern in comparison with NB1691.Luc, as well as a different distribution of vasculature and W765-BG agents. FIG. 4K shows the W765-BG whole body distribution. FIG. 4L shows the merged NGP.Luc images. FIG. 4M shows a tumor node. FIG. 4N shows a vasculature image with the imaging agent having a high signal surrounding the tumor region. The tumor to background ratio reached 1.9. FIG. 4O shows that the W765-BG agent was taken up by the tumor and the tumor to background ratio reached 2.8. FIG. 4P shows optical images of the tumor node, vasculature agent, and W765-BG agent signals were overlaid. The distribution of W765-BG was different than the vasculature agent. W765-BG agent was mainly in the tumor region, while the vasculature agent was in the tumor and kidney regions. FIG. 4Q shows that W765-BG was concentrated in the tumor region at this stage. FIG. 4R shows merged multi-energy images which illustrates the relationship between the anatomy, the disease, and the imaging agents.

FIG. 5 shows the late stage disease, organ, and pathological image. FIG. 5A shows a color image of tumor bearing animal. FIG. 5B shows that a majority of the vasculature agent was located in the kidneys at this disease stage. FIG. 5C shows that W765-BG is located only in the tumor region. FIG. 5D shows a luciferase image with uneven signal distribution in the tumor region. FIG. 5E shows a color image of the organ layout. FIG. 5F shows a merged X-ray and vasculature image that confirms the whole body result that this agent was located in the kidneys. FIG. 5G shows that the W765-BG signal was from the liver, spleen and tumor. FIG. 5H shows a merged organ image with the imaging agents distributed into different organs. Pathology confirmed that the organs with imaging agents were the tumor (FIG. 5I), muscle (FIG. 5J), liver (FIG. 5K), kidney (FIG. 5L), and spleen (FIG. 5M).

FIG. 6 shows the statistical comparison of injection time (FIG. 6A) and cell line (FIG. 6B) differences. FIG. 6A shows the tumor to background ratio differences at different imaging time points. FIG. 6B shows the uptake differences between the cell lines and the W765-BG imaging agent.

### DETAILED DESCRIPTION OF THE INVENTION

It will be readily apparent to one skilled in the art that various embodiments and modifications may be made in the invention disclosed herein without departing from the scope and spirit of the invention.

### I. DEFINITIONS

In any embodiment herein, any R group (R₁, R₂, R₃, R₄, and/or R₅) may be further defined as alkyl_{(C=1-8)}, such as methyl, ethyl, n-propyl, or isopropyl. Any R group may comprise an alkenyl_{(C=1-8)} group, such as allyl. Any R group may comprise a substituted or unsubstituted aralkyl_{(C=1-8)} group, such as benzyl or 2-furanylmethyl.

In any embodiment herein regarding alkyl_{(C=1-8)}, aryl_{(C=1-8)} and aralkyl_{(C=1-8)} groups (e.g., alkyl_{(C=1-8)}, alkyl_{(C=1-8)} sulfonate, alkyl_{(C=1-8)} halide, aryl_{(C=1-8)} sulfonate, aralkyl_{(C=1-8)}, etc.), it is specifically contemplated that the number of carbons may be 1, 2, 3, 4, 5, 6, 7, or 8, or any range derivable therein. It is also specifically contemplated that any particular number of carbon atoms may be excluded from any of these definitions.

As used herein, “halide” means independently —F, —Cl, —Br or —I and “sulfonyl” means —SO₂—.

The term “alkyl,” when used without the “substituted” modifier, refers to a non-aromatic monovalent group, having a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group, having a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃. In certain embodiments, “lower alkyl” groups are contemplated, wherein the total number of carbon atoms in the lower alkyl group is 6 or less.

The term “alkenyl” when used without the “substituted” modifier refers to a monovalent group, having a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group, having a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “aryl,” when used without the “substituted” modifier, refers to a monovalent group, having an aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group, having an aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “aralkyl” when used without the “substituted” modifier refers to the monovalent group -alkanediyl-aryl, in which the terms alkanediyl and aryl are each used in a manner consistent with the definitions provided herein. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are (3-chlorophenyl)-methyl, 2-oxo-2-phenyl-ethyl (phenyl-carbonylmethyl), 2-chloro-2-phenyl-ethyl and 2-methylfuranyl.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCH(F)— and —C≡CCH(Cl)— are non-limiting examples of substituted alkynediyl groups.

The terms “alkyl sulfonate” and “aryl sulfonate” refer to compounds having the structure —OSO₂R, wherein R is alkyl or aryl, as defined above, including substituted versions thereof. Non-limiting examples of alkyl sulfonates and aryl sulfonates include mesylate, triflate, tosylate and besylate. In certain embodiments, mesylates are excluded from compounds of the present invention.

As used herein, “protecting group” refers to a moiety attached to a functional group to prevent an otherwise unwanted reaction of that functional group. The term “functional group” generally refers to how persons of skill in the art classify chemically reactive groups. Examples of functional groups include hydroxyl, amine, sulfhydryl, amide, carboxylic acid, ester, carbonyl, etc. Protecting groups are well-known to those of skill in the art. Non-limiting exemplary protecting groups fall into categories such as hydroxy protecting groups, amino protecting groups, sulfhydryl protecting groups and carbonyl protecting groups. Such protecting groups, including examples of their installation and removal, may be found in Greene and Wuts (1999), incorporated herein by reference in its entirety. The starting materials, products and intermediates described herein are also contemplated as protected by one or more protecting groups—that is, the present invention contemplates such compounds in their “protected form,” wherein at least one functional group is protected by a protecting group.

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974 Recommendations. Thus, in certain aspects, compounds of the present invention may comprise S- or R-configurations at particular carbon centers.

Persons of ordinary skill in the art will be familiar with methods of purifying compounds of the present invention. One of ordinary skill in the art will understand that compounds of the present invention can generally be purified at any step, including the purification of intermediates as well as purification of the final products. Purification procedures include, for example, silica gel column chromatography, HPLC, or crystallization. In particular embodiments, trituration is employed. In certain embodiments, solvent extraction is employed.

Modifications or derivatives of the compounds disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically-modified compound that still retains the desired effects of the compound prior to the chemical modification. Using W765-BG as an example, a “W765-BG derivative” refers to a chemically modified W765-BG that still retains the desired effects of the parent W765-BG prior to its chemical modification. Such effects may be enhanced (e.g., slightly more effective, twice as effective, etc.) or diminished (e.g., slightly less effective, 2-fold less effective, etc.) relative to the parent W765-BG, but may still be considered a W765-BG derivative. Such derivatives may have the addition, removal, or substitution of one or more chemical moieties on the parent molecule. Non-limiting examples of the types of modifications that can be made to the compounds and structures disclosed herein include the addition or removal of lower unsubstituted alkyls such as methyl, ethyl, propyl, or substituted lower alkyls such as hydroxymethyl or aminomethyl groups; carboxyl groups and carbonyl groups; hydroxyls; nitro, amino, amide, imide, and azo groups; sulfate, sulfonate, sulfono, sulfhydryl, sulfenyl, sulfonyl, sulfoxido, sulfonamide, phosphate, phosphono, phosphoryl groups, and halide substituents. Additional modifications can include an addition or a deletion of one or more atoms of the atomic framework, for example, substitution of an ethyl by a propyl, or substitution of a phenyl by a larger or smaller aromatic group. Alternatively, in a cyclic or bicyclic structure, heteroatoms such as N, S, or O can be substituted into the structure instead of a carbon atom.

Salts of any of the compounds of the present invention are also contemplated. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts, such as alkylammonium salts. Salts include, but are not limited to, sodium, lithium, potassium, amines, tartrates, citrates, hydrohalides and phosphates.

Hydrates of compounds of the present invention are also contemplated. The term “hydrate” when used as a modifier to a compound means that the compound has less than one (e.g., hemihydrate), one (e.g., monohydrate), or more than one (e.g., dihydrate) water molecules associated with each compound, such as in solid forms of the compound.

It is contemplated that any embodiment discussed in this specification can be implemented with respect to any method, compound, or composition of the invention, and vice versa. Furthermore, compounds and compositions of the invention can be used to achieve methods of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### II. DISCUSSION OF GENERAL EMBODIMENTS

Currently, MIBG based tumor imaging plays a critical role in the clinical staging and evaluation of therapeutic responses for neuroblastoma. MIBG can be selectively concentrated in more than 90% of neuroblastomas (Maris et al., 2007), and remains stable while circulating throughout the body (Papaioannou and McHugh, 2005). However, the extent of radiation exposure while using this agent in young patients is significant enough to cause secondary cancers in some cases. Therefore, it is necessary to develop an NIR imaging agent with the same functional characteristics as MIBG but without the use of radiation. In recent years, red-infrared excitable compounds and near-infrared excitable compounds have been used for molecular imaging due to the longer wavelengths at which they are detected to increase signal-to-background ratios. Cyanine dyes are commonly used as fluorophores for this purpose. The commercially available cyanine dye IRdye800CW was chosen as the NIR fluorescence contrast agent for this study because of its strong NIR signal intensity and its polarity, as the latter may aid in reducing the amount of imaging agent accumulated in the liver.

The targeting moiety of the imaging agent was designed based on MIBG structure. It is known that MIBG is derived from neuron blocking agents. The MIBG structure combines the guanidine group from guanethidine and the benzyl portion from bretylium (Wieland, 1986). Structural alterations at the side chain of MIBG are critical for its binding specificity (Wieland, 1986; Vaidyanathan, 2008; Vaidyanathan, 2001). In general, benzyl ring substitutions are tolerated to a greater level than modifications on the guanidinomethyl functionality. The replacement at the meta or para position on the benzylguanidine ring maintain high affinities for the adrenal medulla (AM). The removal of iodine from MIBG had little effect on benzyl guanidine's accumulation in the adrenal medulla (Wieland, 1986; Vaidyanathan,1992). Therefore, W765-BG was designed based on meta substitution of benzylguanidine.

The iodine functionality at the meta position of MIBG was replaced by an amino group coupled with glycine. One reason for this replacement was that the polar substituent on the aromatic ring may increase the excretion of the agent from normal tissue and enhance the tumor-to-background ratio. The reason for introducing an amino group coupled with glycine was that the resulting compound has a reactive primary amino functionality capable of conjugating with IRdye800CW. Finally, glycine was inserted as a spacer between the active component and the optical reporter to eliminate steric hindrance caused by the fluorophore, which might interfere with the ability of neuroblastoma cells to take up the compound.

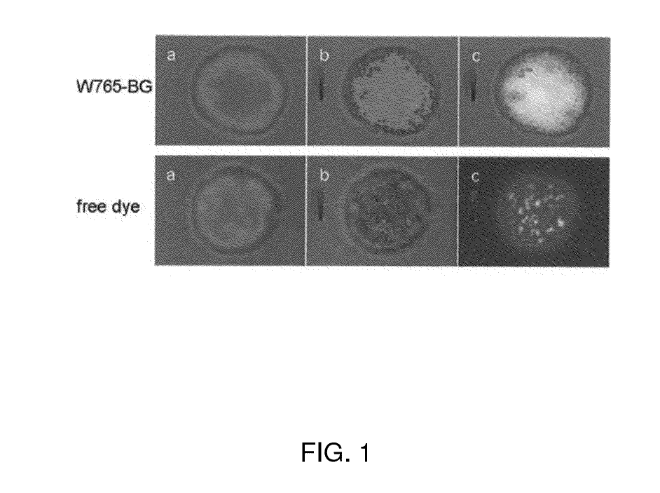

The properties of this newly developed benzyl guanidine analog (W765-BG) were characterized in the in vitro and in vivo studies. The relationship between neuroblastoma cells, as well as the neuroblastoma cells and W765-BG or the free NIR dye was determined in vitro by NIR confocal microscope. A high uptake of optical imaging agent W765-BG in neuroblastoma cell was observed by cell study. FIG. 1 shows a side by side cell binding comparison between these imaging agents at single cell level. As shown in FIG. 1, not only does the imaging agent cross the cell membrane and pass through cytoplasm, but the imaging agent is internalized into the cell nuclei (FIG. 1, top panel, (b) and (c)). There was almost no detectable signal in cell from free dye (FIG. 1, bottom panel, (b) and (c)). This data demonstrates that W765-BG targets neuroblastoma.

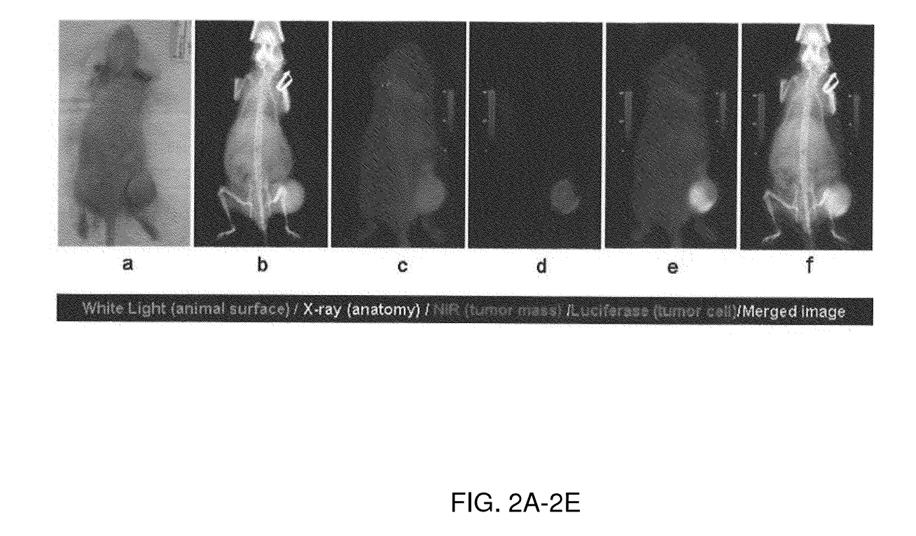

The selectivity of W765-BG was further explored through the in vivo studies using luciferase-positive human neuroblastoma xenografts in mice. FIG. 2 shows four different kinds of images (white light, X-ray, NIR and luciferase) of the same tumor-bearing mouse 48 hours after administration of the imaging agents. The NIR image revealed that W765-BG accumulates in cell inoculation site where the tumor can be visualized by other three images (white light, X-ray and luciferase imaging). Importantly, W765-BG was detected in tumor region only. This result confirms that W765-BG is highly selective for neuroblastoma. Furthermore, it was observed that W765-BG optical signals were retained in the tumor for about 48 hours after injection, after which the optical signal strength gradually diminished. The tumor to background ratios for 24, 48 and 192 hours were 1.95, 1.98, and 1.52, respectively. This permits imaging at prolonged intervals after injection.

W765-BG is specific for neuroblastoma cells and is readily accumulated in neuroblastoma cells. Since, W765-BG can be visualized at the cellular level makes W765-BG a valuable tool for mechanistic studies and in vitro cellular tracking experiments. Furthermore, unlike radiolabeled MIBG, which has to be used immediately after manufacture because of its short half-life, W765-BG can be pre-synthesized and stored over a long period of time, eliminating the time constraint between manufacture and use.

The cell uptake of W765-BG requires more time by neuroblastoma cells suggests this is not receptor-ligand process rather through a metabolic mechanism. The metabolic uptake requires a much longer time for the signals from the agent to be detected in the cells, compared with receptor-ligand or antigen-antibody binding. The cell metabolic conditions also affect the capability of the uptakes. This uptake variance is reflected in the in vivo imaging study results. Using confocal microscopy and collecting more than one channel signal from a single cell slice is important to validate the relationship among multiple signals.

The W765-BG compound is both sensitive and specific for neuroblastoma in the xenograph mouse model. Neuroblastoma specific uptake and visualization using this near-IR dye conjugated analog of benzyl guanidine detected tumors developed from three different cell line. This compound also has a prolonged imaging window after injection suggesting that it accumulates in neuroblastoma tissue and is slowly metabolized or excreted from neuroblastoma cells. Once absorbed, strong tumor specific optical signals were found to persist longer than one week. These findings support the proposition that IR imaging can overcome the limitations of radiolabeled MIBG for neuroblastoma imaging studies as well as provide a more complete understanding of these tumors.

Earlier near-IR imaging has demonstrated feasibility for whole body imaging with a tissue penetration depth sufficient for metastasis and primary tumor detection without ionizing radiation. Radio-labeled MIBG must be synthesized immediately prior to use which is costly and logistically difficult in the clinical setting and severely limits its use for in vitro studies. In contrast W765-BG has a long shelf life facilitating and prolonged imaging window (up to 8-9 days post injection).

W765-BG imaging is specific for tumors in the retroperitoneal area. Normal renal structures did not take up the compound. Limited uptake of W765-BG in the kidney region is important because the adrenal gland is the most common location for neuroblastoma. Furthermore, the W765-BG signal intensity in the liver is much lower than other agents that have been reported thus far. This mouse model did not develop liver or bone metastases which are important locations for clinical applications. Therefore the sensitivity for detecting tumor in those areas remains unknown. However the low W765-BG background signal in the whole body images suggests that uptake in distant sites is tumor specific.

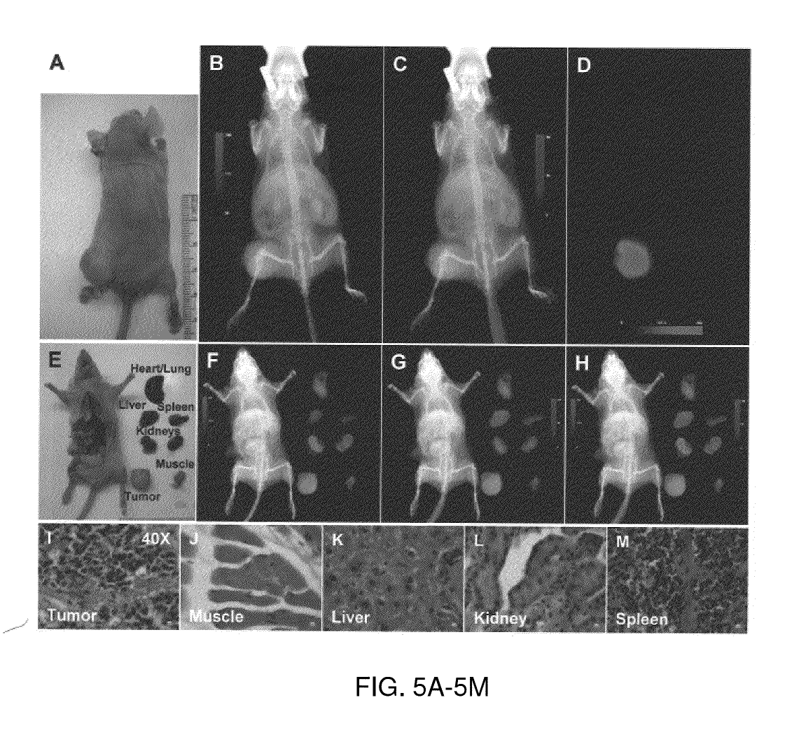

The W765-BG images illustrate quite different tumor growth patterns after inoculation. Neuroblastoma xenografts reflect the heterogeneity of clinical tumors with different growth rates, degrees of vascularity and apoptotic rates. For example, NGP is localized with clear margins whereas the NB 1691 tumors have much more diffuse margins. Dissection and pathological analysis showed necrotic tissue in the low signal intensity part of the tumor. This data suggest that W765-BG is taken up only by viable cells. When combined with RGD 5.5 vascular imaging, the tumor boundaries and regions of neovasculargenesis were better defined (FIG. 5). Further studies with this mode of imaging may help to understand responses to antiangiogenic and other types of targeted therapies in the xenograft setting.

Relative to W765-BG, the lack of homogeneity of luciferase over serial observations suggest that luciferase imaging can be a highly variable measure of tumor size. It is unusual to observe heterogeneous luciferase signal intensity inside a tumor in published luciferase images. It is believed that the reason it was possible to detect regional differences in single tumors was at least partly due to the use of a high resolution and high dynamic range of the CCD in the optical imaging system used in this study. This camera system provides very high image acuity and is sensitive enough to vividly discriminate many levels of signal from background noise without saturating the detector.

The cell uptake of W765-BG requires more time by neuroblastoma cells suggest this is not a receptor-ligand process, but a metabolic mechanism. The metabolic uptake requires a much longer time for the signals from the agent to be detected in the cells, compared with receptor-ligand or antigen-antibody binding. The cell metabolic conditions also affect the capability of the uptakes. This uptake variance is reflected in the in vivo imaging studies. Using confocal microscopy and collecting more than one channel signal from a single cell slice is important to validate the relationship among multiple signals.

It is well known that tumors are heterogeneous. The luciferase images illustrate the different tumor growth patterns after inoculation. Even with two lines of cells that are derived from the same disease, one is localized with clear margins and the other is diffused without margins. These types of imaging findings may be used in clinical decisions regarding tumor staging and treatment. The localized tumor with clear margins may be treated with surgery and/or radiation. On the other hand, the diffused tumor may not be treated with surgical methods because it may be difficult to achieve a clear margin. The high interstitial pressure in the solid tumor may confound chemotherapy because the agents cannot penetrate into the tumor mass. The images provided herein illustrate that such solid tumors may even prevent a small peptide, like RGD, from penetrating into the tumor mass. The other possibility is that this tri-peptide agent does not match this disease at this stage.

The current disclosure provides a novel approach to imaging neuroblastoma tumors using an analogue of MIBG. It was demonstrated that tumor specific uptake was done with a very low background. Multi-agent and multi-wavelength optical imaging used helped to define the interactions among tumor cells, tumor vasculature, and tumor-specific imaging agents. Near-IR optical imaging of neuroblastoma using the imaging agent disclosed herein provides several clinical uses and advantages over standard I¹²³ MIBG imaging. These properties open new possibilities in both in vitro and in vivo studies.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods and Materials

Cell lines. Human neuroblastoma cancer cell lines NB1691.1uc and SYFY.luc were cultured in Dulbecco's Modified Eagle's Medium supplemented with high glucose and F12 nutrient (DMEM/F12, Invitrogen, Carlsbad, Calif.) with 10% fetal bovine serum (Hyclone, Logan, Utah) in a humidified incubator maintained at 37° C. with 5% CO₂.

Tumor xenografts: Four- to six-week-old female nude mice (18-22 g) (Taconic, Hudson, N.Y.) were housed and fed with sterilized pellet chow and sterilized water. Animals were maintained in a pathogen-free mouse colony. Tumor cells near confluence were harvested by incubation with 0.05% trypsin-EDTA. Cells were pelleted by centrifugation at 130×g for 5 min and resuspended in sterile phosphate-buffered saline (PBS). Approximately 1 million cells were implanted subcutaneously into the hind leg region of the mice. A total of 15 mice was used in this study.

Confocal microscope imaging. Cells were harvested from culture and incubated overnight at 37° C. with W765-BG imaging agent or NIR dye at final concentrations of 50 μmolar. Cells were washed in PBS, then incubated for 15 min at 4° C. with Sytox green (Molecular Probes) in 95% ethanol to fix the cells and stain cell nuclei. Stained cells were then transferred to a slide and mounted for microscopic examination. Images were recorded using an Olympus confocal microscope (Model: Fluorview 1000, Olympus America, Center Valley, Pa., USA). The microscope was equipped with excitation (Ex) light source and emission (Em) filters to detect and separate W765-BG or NIR dye (ex/em 765/810 nm) and cell nuclei (ex/em 488/510 nm) signals. In the microscopic images, signal intensities were recorded from one slice of 23 cell z-stacks with 0.5 micrometers gaps. Sytox green and W765-BG or NIR dye signals were pseudo colored into green (emission at 510 nm) and red (emission at 810 nm), respectively.

Animal imaging. Tumors were visualized by intraperitoneal injection of 3 mg of VivoGlo Luciferin (Promega, Madison Wis.) before the imaging session, and pseudo into cyan. Ten nanomoles of W765-BG and 3 nanomoles of the vascular agent RGD-Cy5.5 were injected i.v. into the tumor-bearing mice prior to the imaging study. The animals were imaged 1 to 9 days after the injection using the Kodak In-Vivo Multispectral System FX (Carestream Health Molecular Imaging, New Haven, Conn.). Vasculature images were recorded at wavelengths of ex/em 650/700 nm for RDG-Cy5.5 and pseudo colored into red. The W765-BG signal was recorded at wavelengths of ex/em 770/830 nm and pseudo colored into green. Tumor cells, vasculature, and W765-BG signals were precisely overlaid with anatomical X-ray images.

Chemicals. Boc-glycine-OSu was purchased from Chem-Impex International (Wood Dale, Ill.). 3-aminobenzyl alcohol, N,N-diisopropylethylamine (DIPEA), triethylsilane (TES), 1,3-Bis(tert-butoxycarbonyl)guandine, triphenylphosphine (TPP), and diidsopropyl azo-dicarboxylate (DIAD) were purchased from Sigma-Aldrich (St. Louis, Mo.). Trifluoroacetic acid (TFA) and all other reagents and solvents were purchased from VWR (San Dimas, Calif.). IRdye800cw and IRdye800cw carboxylate were purchased from Li-Cor (Lincoln, Neb.).

Compound Analyses. Analytical high-performance liquid chromatography (HPLC) was performed on an Agilent 1100 HPLC system equipped with a Varian reverse phase C-18 analytical column (R0086200CG) at a flow rate of 1 mL/min. Samples were eluted with H₂O/acetonitrile containing 0.1% TFA with three different linear gradients (A: 0-20% in 10 min; B: 0-40% in 30 min; C: 10-80% in 30 min.). Preparative HPLC was performed on a Varian Prostar 210 HPLC equipped with a 25×2.5-cm Varian reverse phase C-18 preparative column (R0080220CB). Matrix-assisted laser desorption ionization mass spectrometry (MALDI) and electrospray ionization mass spectrometry (ESI) were performed by the Protein Chemistry Core Laboratory at Baylor College of Medicine. Nuclear magnetic resonance (NMR) was performed by CCSG Shared Resources at M.D. Anderson Cancer Center.

### Example 2

### Synthesis of W765-BG

The reaction scheme and structure of W765-BG is shown in Scheme 1. The synthesis procedure was carried out in four steps. Compound 1 (3-aminobenzyl alcohol) was reacted with Boc-Gly-OSu under basic conditions to form 3-(Boc-Gly-amino)benzyl alcohol (Compound 2). By means of the Mitzunobu protocol (Dodd and Kozikowski, 1994), compound 2 was converted to 1,3-bis(tert-butyloxycarbonyl)-2-(3-(Boc-Gly-amino)benzyl)guanidine (compound 3) by treatment with N,N-bis-Boc-guandine, triphenylphosphine, and diidsopropyl azo-dicarboxylate. Three Boc-protecting groups on compound 3 were removed under acidic conditions, resulting in compound 4. This compound was conjugated to IRdye800cw through the α-amino functional group of glycine to result in the NIR optical imaging agent W765-BG.

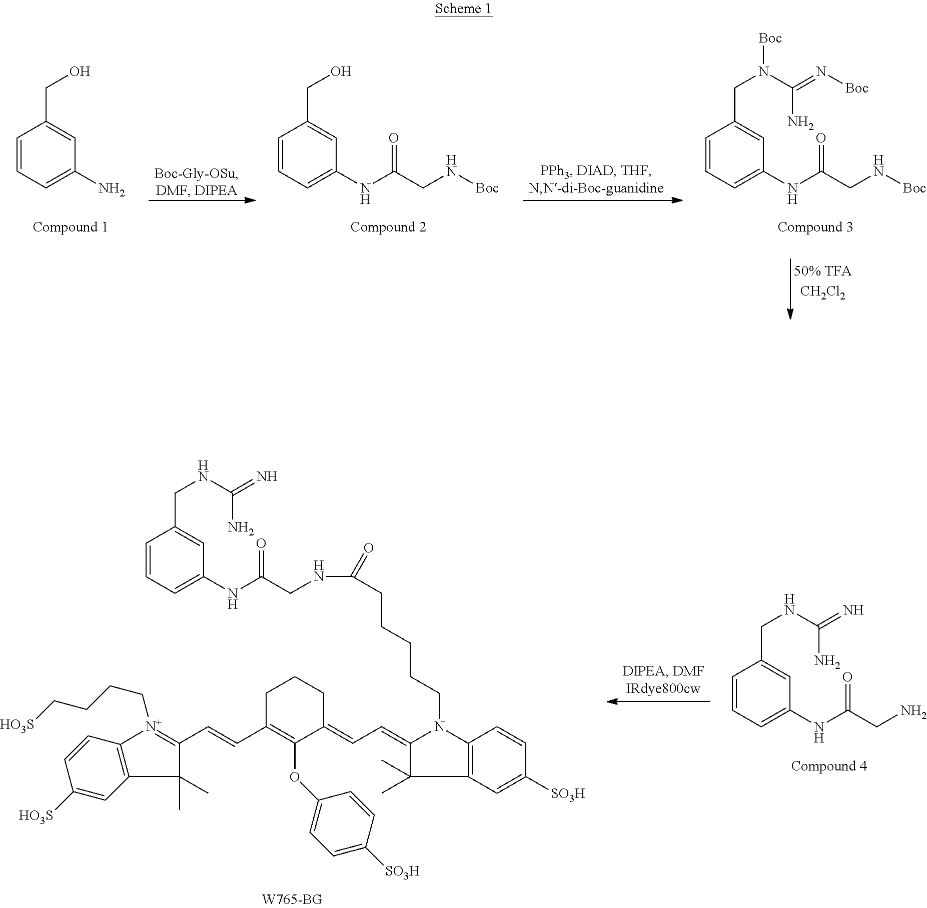 [SMILES: Nc1cccc(CO)c1 ; CC(C)(C)OC(=O)NCC(=O)Nc1cccc(CO)c1 ; CC(C)(C)OC(=O)/N=C(\N)N(Cc1cccc(NC(=O)CNC(=O)OC(C)(C)C)c1)C(=O)OC(C)(C)C ; CC1(C)C(/C=C/C2=C(Oc3ccc(S(=O)(=O)O)cc3)C(=C/C=C3/N(CCCCCC(=O)NCC(=O)Nc4cccc(CNC(=N)N)c4)c4ccc(S(=O)(=O)O)cc4C3(C)C)/CCC2)=[N+](CCCCS(=O)(=O)O)c2ccc(S(=O)(=O)O)cc21 ; N=C(N)NCc1cccc(NC(=O)CN)c1]

Synthesis of 3-(Boc-Gly-amino)benzyl alcohol (Boc-Gly-NH-Bn-OH) (Compound 2): 3-aminobenzyl alcohol (123 mg, 1 mmol) and Boc-Gly-OSu (272 mg, 1 mmol) were dissolved in 5 mL of DMF. DIPEA (0.2 mL) was added to the solution, and the mixture was stirred overnight at room temperature. After evaporation of the solvents under vacuum, the residue was dissolved in ethyl acetate, washed with 5% NaHCO₃, 2% KHSO₄, and brine. Solvents were removed under vacuum. The solid was further purified by reverse phase HPLC with water and acetonitrile as eluent, and dried by lyophilization to yield Compound 2, which was validated by NMR and analytic HPLC. 1H NMR (CDCl₃): 1.49 (s, 9H), 3.93 (s, 2H), 4.65 (s, 2H), 5.45 (b, 1H), 7.07 (d, 1H), 7.25-7.30 (m, 2H), 7.41 (d, 1H), 8.46 (b, 1H); HPLC (gradient C) retention time 10.98 min.

Synthesis of 1,3-bis(tert-butyloxycarbonyl)-2-(3-(Boc-Gly-amino)benzyl)guanidine [Boc-Gly-NH-Bn-G(Boc)₂] (Compound 3): Compound 2 (140 mg, 0.5 mmol), 1,3-Bis(tert-butoxycarbonyl)guandine (259 mg, 1 mmol), and TPP (275 mg, 1.1 mmol) were dissolved in 5 mL of THF. DIAD (203 mg, 1 mmol) was added dropwise to the solution, and the mixture was stirred overnight at room temperature. The THF was evaporated under vacuum, and the residue was purified by flash chromatography using ethyl acetate/hexane as eluent, to yield Compound 3, which was validated by NMR, Mass and analytic HPLC. 1H NMR (CDCl₃): 1.39 (s, 9H), 1.49 (s, 18H), 3.93 (s, 2H), 5.15 (s, 2H), 5.26 (b, 1H), 6.94 (d, 1H), 7.21-7.27 (m, 2H), 7.46 (d, 1H), 8.17 (b, 1H), 9.51 (b, 1H); Mass for C₂₅H₃₉N₅O₇ calculated. [M]⁺ 521.28; found 522.2; HPLC (gradient B) retention time 7.09 min.

Synthesis of 2-(3-(Gly-amino)benzyl)-guanidine (Gly-NH-BG) (Compound 4): Compound 3 (130 mg, 0.25 mmol) was dissolved in 2 mL of 50% TFA in dichloromethane for 1 hr at room temperature. The solvent was evaporated under vacuum, and the residue was purified by reverse phase HPLC with water and acetonitrile containing 0.1% TFA as eluent. The sample was lyophilized to yield Compound 4, which was validated by NMR, Mass and analytic HPLC. 1H NMR (DMSO): 3.78 (s, 2H), 4.37 (d, 2H), 7.02 (d, 1H), 7.33-7.39(m, 3H), 8.12-8.14(m, 4H), 10.51(s, 1H); Mass for C₁₀H₁₅N₅O calculated. [M]⁺ 221.13; found 222.1; HPLC (gradient A) retention time 6.68 min.

Synthesis of 2-(3-(IRdye800-Gly-amino)-benzyl)guanidine (W765-BG) (Compound 5): Conjugation of IRdye800cw (5 mg, 0.004 mmol) to the amino group of Gly in 2-(3-(Gly-amino)benzyl)guanidine (2.33 mg, 0.01 mmol), was carried out in a DIPEA/DMF(1/9) solution for 2 hr at 4° C. The solvent was removed under vacuum and the residue was washed with ether several times. The solid was purified by reverse phase HPLC and dried by lyophilization to yield W765-BG. The compound was validated by Mass and analytic HPLC. MALDI for C₅₆H₆₈N₇O₁₅S₄⁺ calculated [M]⁺ 1206.37, found 1206.36; HPLC (gradient B) retention time 19.86 min.

### Example 3

### Imaging using W765-BG

Imaging of W765-BG in neuroblastoma cells. To evaluate W765-BG as a potential specific molecule, its ability to uptakes by neuroblastoma cells was examined in vitro by NIR confocal microscopy. NPG.Luc cells were treated with W765-BG or free dye overnight at 37° C., after which they were washed, fixed, and visualized by confocal microscopy. W765-BG was detected inside the cells (FIG. 1, top panel), while there was almost no detectable signal in cells treated with the free dye (IRDye800CW carboxylate) alone (FIG. 1, bottom panel). This data confirms that W765-BG is taken up by neuroblastoma cells. The merged confocal images clearly demonstrate that W765-BG is located inside the cell and in the nuclear compartment.

Imaging of W765-BG in human tumor xenografts in mice. To further demonstrate the feasibility of imaging neuroblastomas using W765-BG, luciferase-positive tumor cells (NGP.Luc) were implanted subcutaneously into the hind region of mice. Tumor-bearing mice received an intraperitoneal injection of Luciferin to visualize the tumor cells, followed by an intravenous injection of W765-BG from the tail vein. Whole body images were collected at 24 h intervals over the course of eight days. Tumors were visualized by white light, X-ray and luciferase imaging (FIG. 2). W765-BG accumulated in the tumor (FIG. 2C), as demonstrated by NIR imaging. When merging the optical signal of W765-BG with luciferase and anatomic X-ray images (FIG. 2E-2F), their precise overlay confirms the specificity of W765-BG.

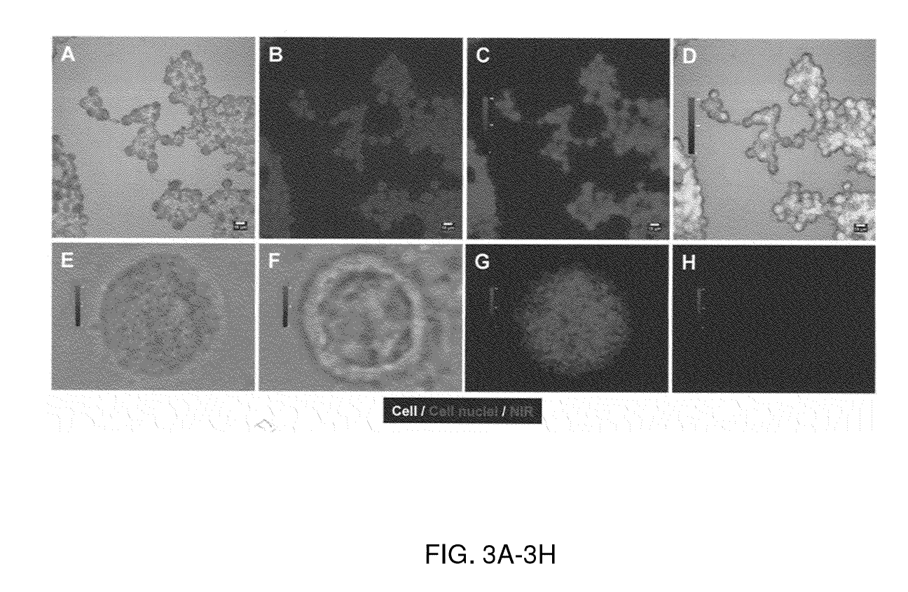

In vitro cell uptake imaging agent. Imaging agent uptake by the cells was recorded by confocal microscopy. The cell population view is shown in FIG. 3 (A to D). The images show cell morphology (FIG. 3A), cell nuclei (FIG. 3B), and W765-BG uptakes by cells (FIG. 3C). The merged image (FIG. 3D) shows that W765-BG was internalized into cells, and co-located with nuclei (yellow color). FIG. 3E to FIG. 3H shows a side-by-side confocal imaging study to compare W765-BG and NIR dye uptake at the single-cell level. The merged morphological and NIR images show that the W765-BG signals were coming from the cell (FIG. 3E), while there was almost no detectable signal in the cells that were incubated with NIR dye in the same imaging setting (FIG. 3F). Those results were validated by non-overlaid NIR images (FIG. 3G and FIG. 3H).

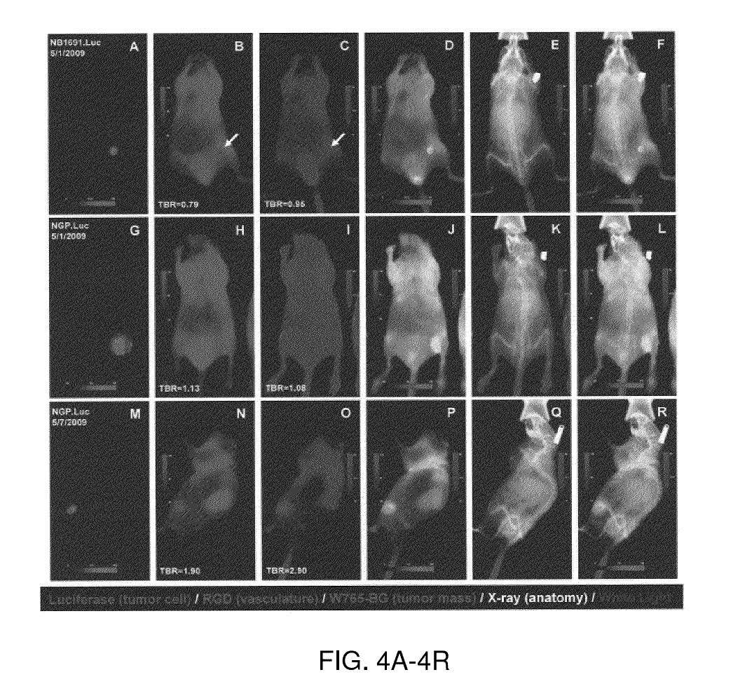

In vivo imaging. Luciferase positive tumor cell could be detected as early as 4 days after inoculation (data not show). Different tumor growth patterns were detected at 7 days post inoculation. NB1691.Luc cells formed a localized node with a clear margin (FIG. 4A). The vasculature image shows a dark centralized region (arrow in FIG. 4B) in the same area. The signal-to-background ratio (TBR) of 0.79 quantitatively demonstrates the lack of vasculature agent in this region. A similar effect was observed in the W765-BG image, with a TBR=0.95 (arrow in FIG. 4C). The merged image shows that the tumor mass fits into this low signal region (FIG. 4D). The anatomic image shows the W765-BG whole body distribution (FIG. 4E). FIG. 4F shows the relationship among the tumor cell, vasculature imaging agent, tumor imaging agent and anatomic structure.

In contrast, the NGP.Luc tumor was rather diffuse without a clear margin in the early tumor growth phase (FIG. 4G). The tumor vasculature developed better than in the NB1691.Luc tumor. This resulted in a higher TBR of 1.13 from the RGD-Cy5.5 agent (FIG. 4H). The diffused tumor growth pattern also limited the W765-BG imaging findings in this stage (FIG. 4I). Once again, the merged images show the location of the tumor cells and the vasculature status (FIG. 4J to FIG. 4L). One week later, the tumor cells became more concentrated in one location (FIG. 4M). The vasculature surrounding the tumor node was well formed (FIG. 4N) and the TBR of the vasculature agent increased to 1.90. The signal intensity of W765-BG was significantly higher in the tumor region than the background and the TBR increased to 2.90 (FIG. 4O). The two imaging agents were distributed differently in the body (FIG. 4P). The vasculature imaging agent RGD was in the kidney and the periphery of the tumor, while W765-BG was in the tumor. Merged X-ray and W765-BG images confirmed the tumor size, location and W765-BG signal intensity (FIG. 4Q). Finally, FIG. 4R vividly shows the mouse anatomic structure, tumor location, and the distribution of two imaging agents.

Late stage tumor, organ image and pathological analysis. Late stage tumor mass and organ imaging were performed after the injection of additional imaging agent (FIG. 5A-H). The color photograph (FIG. 5A) shows the tumor on the left hind leg of the animal. The majority of the vasculature agent signal was concentrated in the kidney region (FIG. 5B), while W765-BG signals were non-uniformly distributed in the tumor (FIG. 5C). A luciferase image of tumor cells also shows uneven signals in the tumor region (FIG. 5D). The dissected animal and organ layout is shown in FIG. 5E. The organ images show the vasculature agent localized in the kidney (FIG. 5F), confirming the whole body imaging results. The dissected organ images show W765-BG in the tumor, liver and spleen (FIG. 5G). The merged image shows the different organ distribution of the two agents (FIG. 5H). Gross necropsy confirmed the necrosis in the center of this tumor. H&E stain confirmed the identification of tumor (FIG. 5I), muscle (FIG. 5J), liver (FIG. 5K), kidney (FIG. 5L), and spleen (FIG. 5M) at pathological levels.

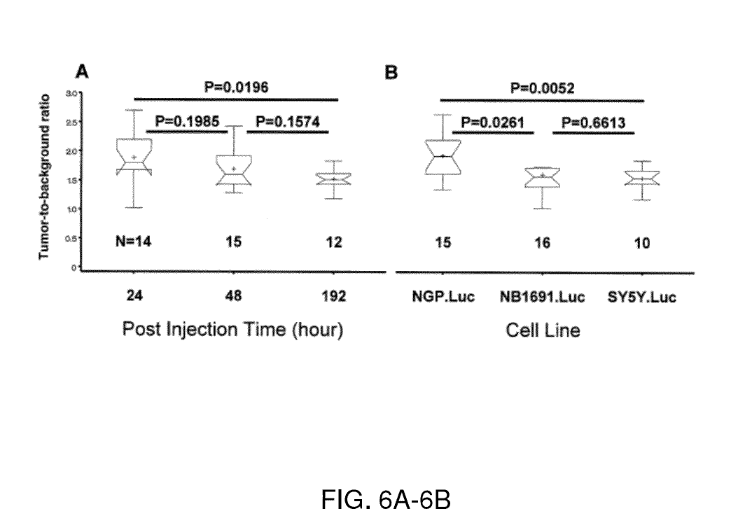

Imaging window and cell type difference. The tumor to background ratio (TBR) was used to determine the optimal imaging time and cell type variance for W765-BG. The TBR is statistically higher at 24 hours after the injection compared with 192 hours (p=0.0196). There was no statistically significant difference in TBR between 24 and 48 hours (P=0.1985) or between 48 and 192 hours (P=0.1574) after injection of this agent in the imaging studies. The results suggested a wide imaging window to achieve consistent data. The data variation also decreased in the 192-hour imaging point compared with the 24-hour. There was a statistically significant difference between the 24 and 192 hours images (FIG. 6A).

As shown in FIG. 6B, significant differences were found among the three cell lines. NGP.Luc cells had a significantly higher uptake capability than NB1691 .Luc (p=0.0261) and SYSY.Luc (p=0.0052). There was no statistical difference between NB1691.Luc and SYSY.Luc (p=0.6613). The best cell line for specific agent binding was the NGP.Luc cells (FIG. 6B).

### Example 4

### Imaging Agents

In some examples, the meta-functionalized benzyl guanidine, is linked to the non-radioactive dye through a general linker moiety. This general linker moiety is composed of a spacer moiety and two connecting functionalities which are chemically bonded to the meta-functionalized benzyl guanidine and the dye. In specific examples, the chemical bond connecting the meta-functionalized benzyl guanidine and the linker moiety is an amide, amine, ester, ether, thioester, a carbon-carbon single bond, a carbon-carbon double bond, or a carbon-carbon triple bond. In specific examples, the spacer moiety of the liker is a functionalized alkyl_{(C=1-8)}, alkenyl_{(C=1-8)}, or an aralkyl_{(C=1-8)}, or substituted versions of any of these moieties. In general, the dye is a contrast agent. In a particular example, the dye is a flurophore.

The imaging agent has the following general structure:

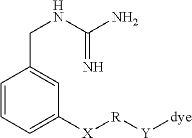

In the general formula for the imaging agent, the general linker moiety is represented by X-R-Y. In some examples, X is chemically bonded to benzyl guanidine through amide, amine, ester, ether, thioether, carbonyl, a carbon-carbon single bond, a carbon-carbon double bond, or a carbon-carbon triple bond. The spacer moiety is represented by R. In some instances, R is alkyl_{(C=1-8)}, alkenyl_{(C=1-8)}, or aralkyl_{(C=1-8)}, or a substituted version of any of these groups. The type of chemical bond used to connect the spacer moiety to the dye may be varied. This chemical bond is represented by Y. In some cases, the spacer moiety is bonded to the dye through an amide or a thioether bond. In specific examples, the dye is IRdye800CW, IRdye800RS, or IRdye700DX.

In general, the imaging agent can be synthesized in four major steps, as shown in Scheme 2. Although Scheme 2 only shows the four major steps, one of ordinary skill in the art would readily recognize that steps such as protections, deprotections and the arrangement of the synthetic process can be modified to arrive at the same imaging agent. For example, in some cases it may be synthetically advantageous to perform step 4 before step 2 or step 3 in order to facilitate purification. In some cases, it may be advantageous to use a protecting agent other than -Boc. There are a wide range variations that can be made to the synthetic route shown in Scheme 2 and still be within the scope of the present invention.

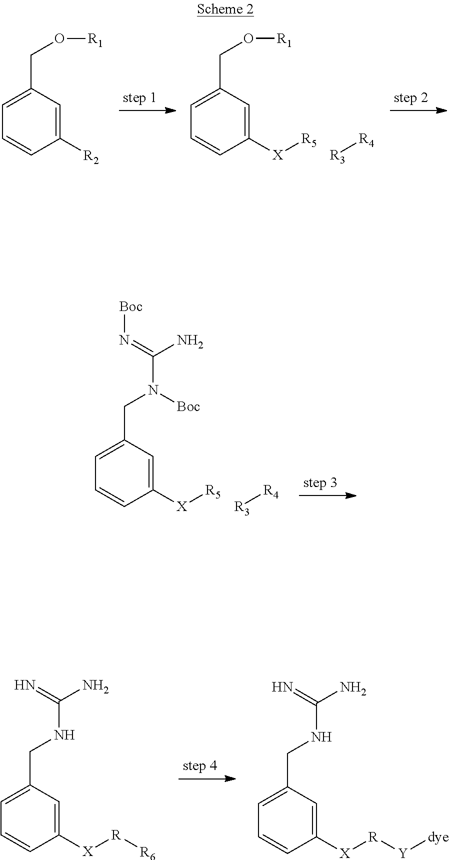

In general, Step 1 comprises coupling the meta-functionalized benzyl alcohol and the linker moiety. The meta-functionalized benzyl alcohol and the linker moiety may be coupled through an amide, an amine, an ester, an ether, thioether, carbon-carbon single bond, carbon-carbon double bond, or a carbon-carbon triple bond.

In the specific example shown below, the meta-functionalized benzyl alcohol is coupled to the linker moiety through an amide bond.

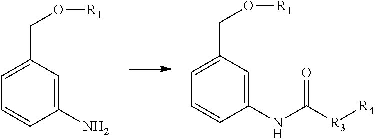

In this specific example, the coupling is accomplished through a two step process. The reaction conditions for the first step include adding N,N′-diisopropylcarbodiimide (DIC) and N-Hydroxysuccinimide (HOSu) to the linker moiety. In this example, the linker moiety is a carboxylic acid represented by the formula R₄—R₃—COOH. The second step of the coupling process includes treating the reaction mixture with a base at room temperature.

In some examples R₁ is hydrogen or trityl (Trt). In specific examples R₃—COOH is glycine, alanine, valine, phenylalanine, leucine, 3-aminopropanoic acid, 4-aminobutanoic acid, 5-aminopentanoic acid, 6-aminohexanoic acid, 7-aminoheptanoic acid, 8-aminooctanoic acid, 4-aminobenzoic acid, 4-mercaptobenzoic acid, 2-mercaptoacetic acid, or 3-mercaptopropanoic acid. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through an amine bond.

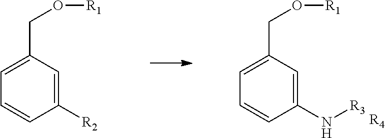

In this specific example, the coupling is accomplished through an one step process. The reaction conditions for this coupling include reacting the meta-halo-functionalized benzyl alcohol with a primary amine represented by the formula R₄—R₃—NH₂ in the presence of a base.

In some examples R₁ is hydrogen or trityl (Trt) and R₂ is a halogen. In specific examples, R₂ is Chlorine or Bromine. In specific examples, R₃—NH₂ is ethane-1,2-diamine, propane-1,3-diamine, pentane-1,5-diamine, hexane-1,6-diamine. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through an ester bond.

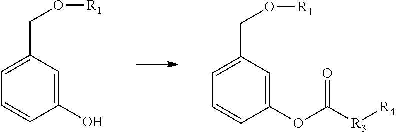

In this specific example, the coupling is accomplished through an one step process. The reaction conditions for this coupling include reacting the meta-hydroxy-functionalized benzyl alcohol with a carboxylic acid represented by the formula R₄—R₃—COOH, N,N′-diisopropylcarbodiimide (DIC) and 4-Dimethylaminopyridine (DMAP) at room temperature.

In some examples R₁ is hydrogen or trityl (Trt). In specific examples, R₃—COOH is glycine, 3-aminopropanoic acid, 4-aminobutanoic acid, 5-aminopentanoic acid, 6-aminohexanoic acid, 7-aminoheptanoic acid, 8-aminooctanoic acid, 4-aminobenzoic acid. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through an ether bond.

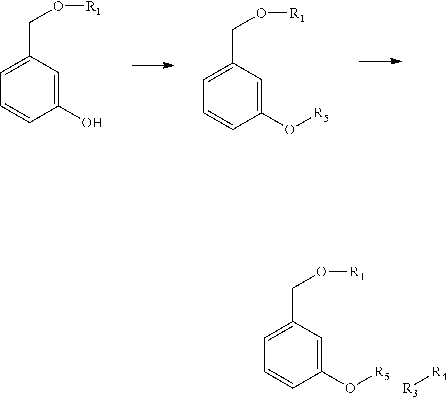

In this specific example, the coupling is accomplished through a multistep process. The first step involves coupling of the meta-hydroxy-functionalized benzyl alcohol with either a cyano-functionalized alkyl halide or a cyano-functionalized alkene in the presence of a base followed by treating the resulting mixture with a borane-tetrahydrofuran complex (BH₃-THF). In specific examples, the cyano-functionalized alkyl halide is BrCH₂CN and the cyano-functionalized alkene is CH₂═CHCN. The resulting primary amine is then coupled to a carboxylic acid represented by the formula R₄—R₃—COOH using N,N′-diisopropylcarbodiimide (DIC) and 4-Dimethylaminopyridine (DMAP) at room temperature followed by treating the resulting reaction mixture with a base.

In some examples R₁ is trityl (Trt). In specific examples, when the cyano-functionalized alkyl halide is BrCH₂CN, R₅ is (CH₂)₂NH₂, and when the cyano-functionalized alkene is CH₂═CHCN, R₅ is —(CH₂)₃NH₂. In some examples, R₃ may or may not be present. When R₃ is not present the coupling agent is N-(9-Fluorenylmethoxycarbonyloxy) succinimide (Fmoc-Osu). In specific examples, R₃—COOH is glycine, alanine, valine, phenylalanine, leucine, 3-aminopropanoic acid, 4-aminobutanoic acid, 5-aminopentanoic acid, 6-aminohexanoic acid, 7-aminoheptanoic acid, or 8-aminooctanoic acid. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through a thioether bond.

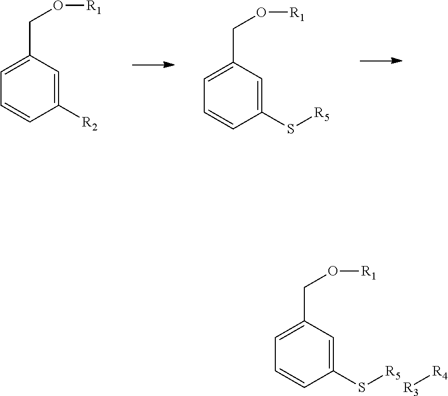

In this specific example, the coupling is accomplished through a two step process. The first step involves coupling of the meta-halo-functionalized benzyl alcohol with a thiol represented by the formula R₅—SH in the presence of a base. In general the R₅ functional group contains a reactive carboxylic acid moiety. The second step involves the reacting the reactive carboxylic acid with an amine represented by the formula R₄—R₃—NH₂, N,N′-diisopropylcarbodiimide (DIC) and N-Hydroxysuccinimide (HOSu).

In some examples R₁ is hydrogen or trityl (Trt) and R₂ is a halogen. In specific examples, R₂ is Chlorine or Bromine. In specific examples, R₅—SH is 4-mercaptobenzoic acid, 2-mercaptoacetic acid or 3-mercaptopropanoic acid. In specific examples, R₃—NH₂ is ethane-1,2-diamine, propane-1,3-diamine, pentane-1,5-diamine or hexane-1,6-diamine. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through a carbon-carbon single bond.

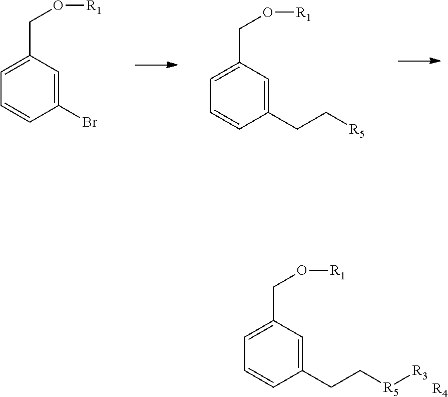

In this specific example, the coupling is accomplished through a two step process. The first step involves coupling of the meta-halo-functionalized benzyl alcohol with a lithium dialkyl copper reagent ((R₅)₂CuLi) in tetrahydrofuran (THF) at −78° C. In general the lithium dialkyl copper reagent comprises a R₅ functional group further comprising a terminal halide. The second step involves the reacting the resulting terminal halide with an amine represented by the formula R₄—R₃—NH₂, in the presence of a Pentamethylcyclopentadienyl)iridium(III) chloride dimer and a base, at reflux.

In some examples R₁ is hydrogen or trityl (Trt). In general, R₅ is an alkyl halide. In specific embodiments, R₅ is bromobutane (—(CH₂)₄Br), or bromoethane (—(CH₂)₂Br). In some examples, R₃—NH₂ is ethane-1,2-diamine, propane-1,3-diamine, pentane-1,5-diamine, or hexane-1,6-diamine. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through a carbon-carbon double bond.

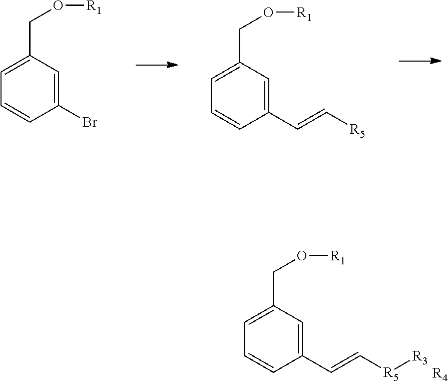

In this specific example, the coupling is accomplished through a two step process. The first step involves coupling of the meta-halo-functionalized benzyl alcohol with an alkene having the general formula R₅—C═CH₂, triphenylphosphine (PPh₃), palladium acetate (Pd(OAc)₂), a base, at about 150° C. The second step involves adding the resulting alkene with a primary amine represented by the formula R₄—R₃—NH₂, N,N′-diisopropylcarbodiimide (DIC) and N-Hydroxysuccinimide (HOSu) to give the meta-functionalized benzyl alcohol coupled to the linker moiety through a carbon-carbon double bond.

In some examples R₁ is trityl (Trt). In general, the formula R₅—CH═CH₂ represents an alkene with a reactive carboxylic acid functionality. In particular examples, R₅—CH═CH₂ is HOOC—CH═CH₂. In specific examples, R₃—NH₂ is ethane-1,2-diamine, propane-1,3-diamine, pentane-1,5-diamine, and hexane-1,6-diamine. In some examples, R₄ is 9-fluorenylmethoxycarbonyl (Fmoc) or tert-butyloxycarbonyl (t-Boc or Boc).

In the specific example shown below, the meta-functionalized benzyl alcohol is coupled to the linker moiety through a carbon-carbon triple bond.

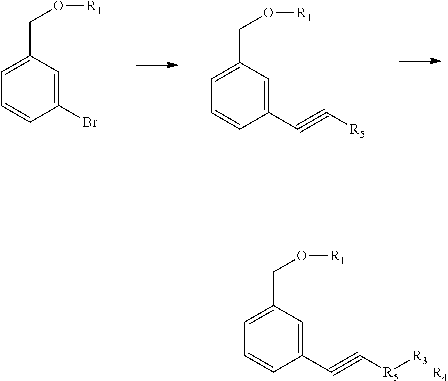

In this specific example, the coupling is accomplished through a two step process. The first step involves coupling of the meta-halo-functionalized benzyl alcohol with an alkyne. This reaction involves the addition of the meta-halo-functionalized benzyl alcohol with an alkyne represented by the formula R₅—C≡CH, pyrrolidine, tetrakis(triphenylphosphine) Palladium (Pd(PPh₃)₄), Copper(I)iodide (CuI), at about 70° C. The second step involves adding the resulting alkyne to a primary amine represented by the formula R₄—R₃—NH₂, a Pentamethylcyclopentadienyl)iridium(III) chloride dimer and a base at reflux.

In some examples R₁ is trityl (Trt). In general, the formula R₅—C≡CH represents a hydroxy functionalized alkyne. In a specific example, the formula R₅—C≡CH represents HOCH₂—C≡CH. In specific examples, R₃—NH₂ is ethane-1,2-diamine, propane-1,3-diamine, pentane-1,5-diamine, or hexane-1,6-diamine. In some examples, R₄ is 9-fluorenylmethoxycarbonyl (Fmoc) or tert-butyloxycarbonyl (t-Boc or Boc).

Generally in Step 2, the meta-functionalized benzyl alcohol is converted to the meta-functionalized benzyl guanidine as shown in the scheme below.

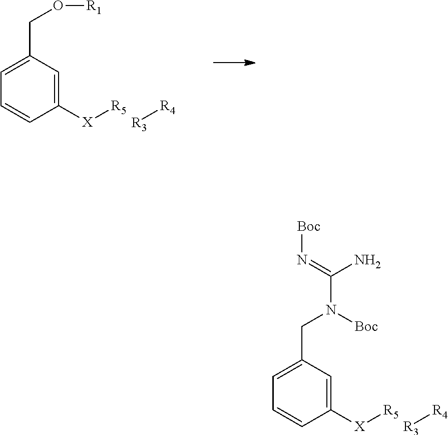

In this specific example, converting the meta-functionalized benzyl alcohol to the meta-functionalized benzyl guanidine may be accomplished in a single step or through a two step process. In specific examples, the meta-functionalized benzyl alcohol is protected and R₁ is a trityl group (Trt). When the meta-functionalized benzyl alcohol is protected with a trityl group, the first step is a deprotection step which removes the trityl group. This step involves treating the protected meta-functionalized benzyl alcohol with 1% trifluoroacetic acid (TFA) in dichloromethane (DCM). When R₁ is hydrogen, this deprotection step is not necessary.

The deprotected meta-functionalized benzyl alcohol is then treated with N,N-bis-Boc-guanidine, triphenylphosphine (TPP), and diisopropyl azodicarboxylate (DIAD) in tetrahydrofuran (THF) to generate the meta-functionalized benzyl guanidine.

In Step 3 of the synthetic process, the protecting groups are removed as shown in the scheme below.

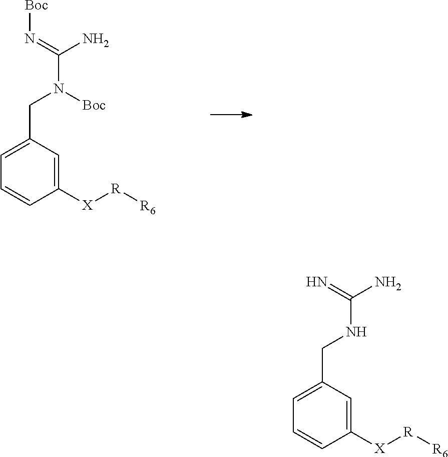

In specific examples, the protecting groups are removed by treating the meta-functionalized benzyl guanidine with 50% trifluoroacetic acid (TFA) in dichloromethane (DCM) for removing Trt and Boc protecting groups. In other examples when the protecting group is Fmoc, the protecting group is removed by treating the meta-functionalized benzyl guanidine with 20% piperidine in dimethylformamide (DMF).

In particular examples, X is —NHCO—, —NH—, —OCO—, —O—, —S—, —CH₂CH₂—, —CH═CH—, or —C≡C—. In general, R is alkyl_{(C=1-8)}, alkenyl_{(C=1-8)}, or aralkyl_{(C=1-8)}, a substituted version of any of these groups, or a substituted and functionalized version of any of these groups. In specific examples, R₆ is —NH₂ or —SH.

In step 4 of the synthetic route, a dye is conjugated to the benzyl guanidine analog as shown in the scheme below.

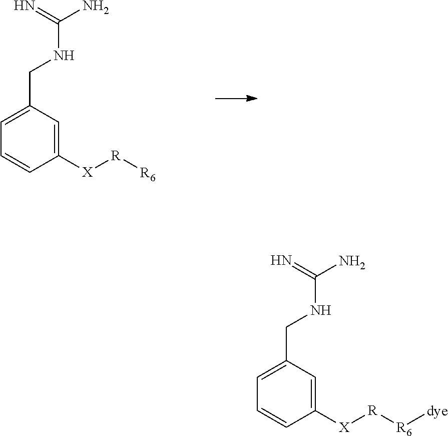

In some examples, the dye is conjugated to the benzyl guanidine analog in one step. In general, this step involves treating the benzyl guanidine analog with a dye, a base at room temperature to give the desired imaging agent.

In particular examples, X is —NHCO—, —NH—, —OCO—, —O—, —S—, —CH₂CH₂—, —CH═CH—, or —C≡C—. In general, R is alkyl_{(C=1-8)}, alkenyl_{(C=1-8)}, or aralkyl_{(C=1-8)}, a substituted version of any of these groups, or a substituted and functionalized version of any of these groups. In specific examples, R₆ is —NH₂ or —SH. In specific examples, the dye is IRdye800CW, IRdye800RS, or IRdye700DX when the benzyl guanidine analog is coupled to the dye through an amino functional group, and the dye is IRDye 800CW Maleimide when the benzyl guanidine analog is coupled to the dye through a thiol functional group.

In a specific example, X is an amide, R is glycine, R₆ is a carboxylic acid functionalized five carbon alkyl chain and the dye is IRdye800CW and the composition has the formula:

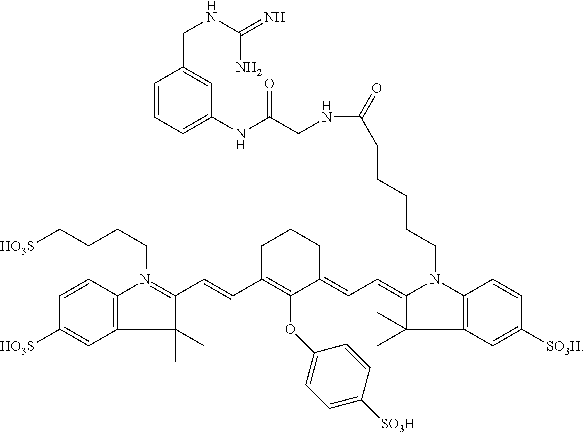 [SMILES: CC1(C)C(/C=C/C2=C(Oc3ccc(S(=O)(=O)O)cc3)C(=C/C=C3/N(CCCCCC(=O)NCC(=O)Nc4cccc(CNC(=N)N)c4)c4ccc(S(=O)(=O)O)cc4C3(C)C)/CCC2)=[N+](CCCCS(=O)(=O)O)c2ccc(S(=O)(=O)O)cc21]

### IV. REFERENCES

The following references, to the extent that they provide exemplary procedural or other details supplementary to those set forth herein, are specifically incorporated herein by reference.

Dodd, D. S., and Kozikowski, A. P. (1994) Tetrahedron Letters 35, 977.

Estorch, M. Eur. J. Nucl. Med. Mol. Imaging, (2006), 33, 246-247.

Henneman, M. M., Bax, J. J., and Van der Wall, E. E. Eur. Heart J., (2007), 28: 922-923.

Howman-Giles, R., Shaw, P. J., Uren, R. F., and Chung, D. K. Semin. Nucl. Med., (2007), 37(4), 286-302.

Kaste, S. C. Pediatr. Radiol., (2009), 39 Suppl 1: S74-79, 1.

Lonergan, G. J., Schwab, C. M., Suarez, E. S., and Carlson, C. L. Radiographics, (2002) 22(4), 911-934.

Mairs, R. J. and Boyd, M., Nuc. Med. Bio., (2008) 35 Supp. 261: S9-20.

Maris, J. M., Hogarty, M. D., Bagatell, R., and Cohn, S. L. Lancet, (2007), 369(9579), 2106-2120.

Papaioannou, G., and McHugh, K. Cancer Imaging, (2005), 5, 116-127.

Park, J. R., Eggert, A., and Caron, H. Pediatr. Clin. North Am., (2008), 55: 97-1 20, x.

Rha, S. E., Byun, J. Y., Jung, S. E., Chun, H. J., Lee, H. G., and Lee, J. M. Radiographics, (2003), 23(1), 29-43.

Robbins, E. R, Pediatr. Blood Cancer, (2008), 51: 453-457.

Rufini, V., Calcagni, M. L., and Baum, R. P. Semin. Nucl. Med. (2006), 36(3), 228-247.

Shirani, J. and Dilsizian, V. Curr. Opin. Biotechnol, (2007), 18, 65-72.

Sisson, J. C., and Shulkin, B. L. Q. J. Nucl. Med., (1999), 43(3), 217-223.

Vaidyanathan, G., Affleck, D. J., Alston, K. L., Welsh, P., and Zalutsky, M. R. (2004) Nucl. Med. Commun. 25(9), 947-955.

Vaidyanathan, G., and Zalutsky, M. R., Bioconjug. Chem., (1992), 3(6), 499-503.

Vaidyanathan, G., Q. J. Nucl. Med. Mol. Imaging, (2008), 52(4), 351-368.

Vaidyanathan, G., Shankar, S., and Zalutsky, M. R. Bioconjug. Chem., (2001), 12(5), 786-797.

Valk, T. W., Frager, M. S., Gross, M. D., Sisson, J. C., Wieland, D. M., Swanson, D. P., Mangner, T. J., and Beierwaltes, W. H. Ann. Intern. Med, (1981), 94(6), 762-767.

Vik, T. A., Pfluger, T., Kadota, R., Castel, V., Tulchinsky, M., Farto, J. C., Heiba, S., Serafini, A., Tumeh, S., Khutoryansky, N., and Jacobson, A. F. Pediatr. Blood Cancer, (2009), 52(7), 784-790.

Wafelman, A. R., Hoefnagel, C. A., Maes, R. A., and Beijnen, J. H., Eur. J. Nucl. Med. (1994), 21(6), 545-559.

Wieland, D. M., Radiopharmaceuticals: Progress and Clinical Perspectives, (1986) 1, 117-153.

Wieland, D. M., Wu, J., Brown, L. E., Mangner, T. J., Swanson, D. P., and Beierwaltes, W. H. J. Nucl. Med., (1980), 21(4), 349-353.

Wong, F. C., and Kim, E. E. Eur. J. Radiol., (2009), 70(2), 205-211.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A composition comprising:
a meta-functionalized benzyl guanidine
a spacer moiety comprising a reactive amino functionality wherein the spacer moiety is chemically bonded to the meta-functionalized benzyl guanidine at the meta position of the benzyl guanidine;
a linker moiety that is chemically coupled to the spacer moiety through the reactive amino functionality of the spacer moiety; and,
a dye that is chemically bonded to the linker moiety.

2. The composition of claim 1 wherein the chemical bond connecting the meta-functionalized benzyl guanidine and the spacer moiety forms a ester, ether, thioether, thioester, amide, a carbon-carbon single bond, a carbon-carbon double bond, or a carbon-carbon triple bond.

3. The composition of claim 2 wherein the spacer moiety further comprises an alkyl_{(C=1-8)}, alkenyl_{(C=1-8)}, or aralkyl_{(C=1-8)}, or a substituted version of any of these groups.

4. The composition of claim 2 wherein the linker moiety is an alkyl_{(C=1-8)}, alkenyl_{(C=1-8)}, or aralkyl_{(C=1-8)}, or a substituted version of any of these groups.

5. The composition of claim 2 wherein the dye is a contrast agent.

6. The compositions of claim 5 wherein the dye is IRdye800CW.

7. The composition of claim 5 wherein the dye is IRdye800RS.

8. The composition of claim 5 wherein the dye is IRdye700DX.

9. A composition having the general formula: 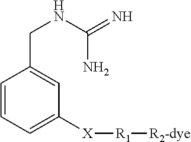
wherein
X is O, CH₂, CH, C, NH, S or C═O;
R₁ has the general formula
R₃-R₄-R₅
wherein R₃ is chemically bonded to X to form an amide, thioester, ester, ether, carbon-carbon bond, carbon-carbon double bond, or a carbon-carbon triple bond,
R₄ is alkyl_{(C=1-8)}, alkenyl_{(C=1-8)}, or aralkyl_{(C=1-8)}, or a substituted version of any of these groups,
R₅ is chemically bonded to R₂ to from an amide, thioester, ester, ether, carbon-carbon bond, carbon-carbon double bond, or a carbon-carbon triple bond,
R₂ is alkyl_{(C=1-8)}, alkenyl_{(C=1-8)}, or aralkyl_{(C=1-8)}, or a substituted version of any of these groups and forms an amino bond with dye; and,
dye is a fluorophore.

10. The composition of claim 9, wherein X is NH.

11. The composition of claim 10, wherein R₃ forms an amide bond.

12. The composition of claim 11, wherein R₄ is alkyl_{(C=1-2)}.

13. The composition of claim 12, wherein R₅ and the R₂ are chemically bonded by an amide bond.

14. The composition of claim 13, wherein the dye is IRdye800CW.

15. The composition of claim 14 having the formula:

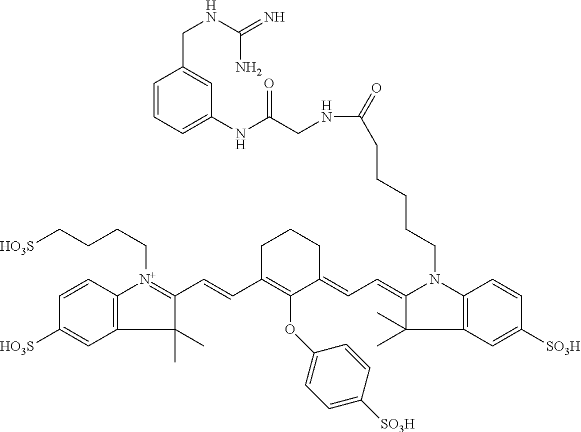 [SMILES: CC1(C)C(/C=C/C2=C(Oc3ccc(S(=O)(=O)O)cc3)C(=C/C=C3/N(CCCCCC(=O)NCC(=O)Nc4cccc(CNC(=N)N)c4)c4ccc(S(=O)(=O)O)cc4C3(C)C)/CCC2)=[N+](CCCCS(=O)(=O)O)c2ccc(S(=O)(=O)O)cc21]

16. A method for imaging neuroblastomas comprising the steps of:
treating a subject with a compound having the general formula: 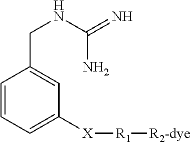
wherein
X is O, CH₂, CH, C, NH, S or C═O;
R₁ is has the general formula
R₃-R₄-R₅
wherein R₃ is chemically bonded to X to form an amide, thioester, ester, ether, carbon-carbon bond, carbon-carbon double bond, or a carbon-carbon triple bond,
R₄ is alkyl_{(C=1-8)}, alkenyl_{(C=1-8)}, or aralkyl_{(C=1-8)}, or a substituted version of any of these groups,
R₅ is chemically bonded to R₂ to from an amide, thioester, ester, ether, carbon-carbon bond, carbon-carbon double bond, or a carbon-carbon triple bond,
R₂ is alkyl_{(C=1-8)}, alkenyl_{(C=1-8)}, or aralkyl_{(C=1-8)}, or a substituted version of any of these groups and forms an amino bond with dye; and,
dye is a fluorophore.

17. The method of claim 16, wherein the dye is IRDye800CW.

18. The method of claim 16 wherein the compound has the formula:

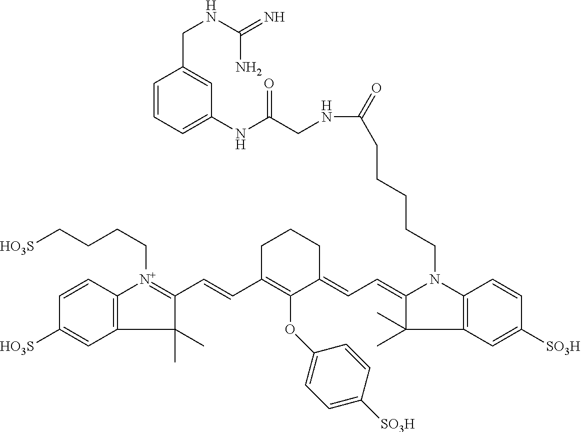 [SMILES: CC1(C)C(/C=C/C2=C(Oc3ccc(S(=O)(=O)O)cc3)C(=C/C=C3/N(CCCCCC(=O)NCC(=O)Nc4cccc(CNC(=N)N)c4)c4ccc(S(=O)(=O)O)cc4C3(C)C)/CCC2)=[N+](CCCCS(=O)(=O)O)c2ccc(S(=O)(=O)O)cc21]

19. The method of claim 18, wherein the compound is non-radioactive.

20. The method of claim 16, wherein the dye is a cyanine dye.
